(19) United States
(12) Patent Application Publication
(10) Pub. No.: US 2026/0188424 A1
(43) Pub. Date: Jul. 2, 2026

(54) SEQUENCE-BASED FRAMEWORK TO DESIGN PEPTIDE-GUIDED DEGRADERS

(71) Applicant: UbiquiTx, New York, NY (US)

(72) Inventors: Garyk BRIXI, New York, NY (US); Pranam CHATTERJEE, New York, NY (US)

(73) Assignee: UbiquiTx, New York, NY (US)

(21) Appl. No.: 19/128,080

(22) PCT Filed: Nov. 7, 2023
(86) PCT No.: PCT/US2023/078949

§ 371 Date: May 7, 2025

Related U.S. Application Data
(60) Provisional application No. 63/423,320, filed on Nov. 7, 2022.

(51) Int. Cl. G16B 20/00; G16B 40/00

(52) U.S. Cl. CPC G16B 20/00; G16B 40/00

(57) ABSTRACT

A Method of generating binding peptide sequences to a target sequence, the method comprising: Receiving, using a processor configured by code executing therein, a data object corresponding to a protein target; Searching, using the data object, a protein interaction database for at least one partner protein to the target protein; Identifying at least one partner protein to the target protein; Providing the at least one partner protein to a computational model configured to output a predicted protein sequence predicted to interact with the target sequence; and Identifying at least one subsequence within the predicted protein sequence that meets a predetermined interaction threshold.

### CROSS REFERENCE TO RELATED APPLICATION

This application claims priority to U.S. Patent Application No. 63/423,320, filed Nov. 7, 2022, which is hereby incorporated by reference in its entirety.

### FIELD OF INVENTION

The present disclosure relates to systems and method for providing a unified, sequence-based framework to design peptide-guided degraders without structural information, wherein such peptide-guided degraders can be used in diagnostic, analytic and therapeutic applications and compositions relating to the same.

### BACKGROUND

Curing malignancies is one of the greatest challenges for the future of human health, and targeted therapeutics have served as potent solutions to this problem. Small molecule inhibitors, specifically, have found significant success in the clinic, but are still limited in their therapeutic potential. Most notably, they are “occupancy-driven,” thus relying on high dosages, and must bind active sites, which either are not present or accessible on classically “undruggable” target proteins. To overcome these limitations, targeted protein degradation (TPD) presents the unique opportunity to bind to intracellular proteins transiently and induce their degradation by hijacking the cell's natural ubiquitin proteasome pathway (UPP).1 As an example, proteolysis-targeting chimeras (PROTACs) and molecular glues employ small molecules that both bind to the target protein and recruit endogenous E3 ubiquitin ligases, enabling ubiquitin transfer to the target protein and subsequent proteasomal degradation. Small molecule-based degraders, however, lack programmability: they require extensive small molecule screening and design at the targeting end, have only been able to leverage a few of the ˜600 E3 ubiquitin ligases, and cannot degrade proteins without accessible binding sites. As a more modular strategy, we and others have recently fused compact protein binders, including FN3s, DARPins, nanobodies, and peptides, to various E3 ubiquitin ligase domains, to enable binding, selective ubiquitination, and intracellular degradation of diverse pathogenic targets of interest. Generating a programmable system for designing these genetically-encoded “ubiquibodies” (uAbs) will represent a more powerful approach for TPD, as compared to more restrictive small molecule-based methods.

Previous approaches for binder design include high-throughput screening and structure-based rational design. More recent computational protein design tools consist of interface predictors, docking software, and inpainting models, which leverage advances in protein structure prediction, such as AlphaFold, to infer new sequences from user-specified structures. These algorithms, such as ProteinMPNN, rely heavily on the existence of either co-crystal complexes or accurate structural predictions of the target protein, thus excluding disordered or unstable proteins, such as transcription factors, which have significant disease implications and are difficult to solve via experimental or computational protein structure determination methods. Recently, language models have been pre-trained on millions of natural protein sequences to generate latent embeddings that grasp relevant physicochemical, functional, and most notably, tertiary structural information. Transfer learning with these models has led to sequence and structure-based prediction of peptide binding sites in a rotationally and translationally invariant manner. Even more interestingly, early results suggest that sequence-based protein transformers can produce novel protein sequences with functional capability.

The following references are herein incorporated by reference as if presented in their respective entireties: Békés, M., Langley, D. R. & Crews, C. M. PROTAC targeted protein degraders: the past is prologue. Nat. Rev. Drug Discov. 21, 181-200 (2022). Schreiber, S. L. The Rise of Molecular Glues. Cell 184, 3-9 (2021). Gao, H., Sun, X. & Rao, Y. PROTAC Technology: Opportunities and Challenges. ACS Med. Chem. Lett. 11, 237-240 (2020). Portnoff, A. D., Stephens, E. A., Varner, J. D. & DeLisa, M. P. Ubiquibodies, synthetic E3 ubiquitin ligases endowed with unnatural substrate specificity for targeted protein silencing. J. Biol. Chem. 289, 7844-7855 (2014). Chatterjee, P. et al. Targeted intracellular degradation of SARS-COV-2 via computationally optimized peptide fusions. Communications Biology 3, 1-8 (2020). Stephens, E. A. et al. Engineering Single Pan-Specific Ubiquibodies for Targeted Degradation of All Forms of Endogenous ERK Protein Kinase. ACS Synth. Biol. 10, 2396-2408 (2021). Lim, S. et al. bioPROTACs as versatile modulators of intracellular therapeutic targets including proliferating cell nuclear antigen (PCNA). Proc. Natl. Acad. Sci. U.S.A. 117, 5791-5800 (2020). Sheehan, J. & Marasco, W. A. Phage and Yeast Display. Microbiol Spectr 3, AID-0028-2014 (2015). Barlow, K. A. et al. Flex ddG: Rosetta Ensemble-Based Estimation of Changes in Protein-Protein Binding Affinity upon Mutation. J. Phys. Chem. B 122, 5389-5399 (2018). Chevalier, A. et al. Massively parallel de novo protein design for targeted therapeutics. Nature 550, 74-79 (2017). Pagadala, N. S., Syed, K. & Tuszynski, J. Software for molecular docking: a review. Biophys. Rev. 9, 91-102 (2017). Abdin, O., Nim, S., Wen, H. & Kim, P. M. PepNN: a deep attention model for the identification of peptide binding sites. Communications Biology 5, 1-10 (2022). Cao, L. et al. Design of protein-binding proteins from the target structure alone. Nature 605, 551-560 (2022). Anishchenko, I. et al. De novo protein design by deep network hallucination. Nature 600, 547-552 (2021). Jumper, J. et al. Highly accurate protein structure prediction with AlphaFold. Nature 596, 583-589 (2021). Dauparas, J. et al. Robust deep learning-based protein sequence design using ProteinMPNN. Science eadd2187 (2022). Varadi, M. et al. AlphaFold Protein Structure Database: massively expanding the structural coverage of protein-sequence space with high-accuracy models. Nucleic Acids Res. 50, D439-D444 (2022). Rives, A. et al. Biological structure and function emerge from scaling unsupervised learning to 250 million protein sequences. Proc. Natl. Acad. Sci. U.S.A. 118, (2021). Madani, A. et al. ProGen: Language Modeling for Protein Generation. bioRxiv 2020.03.07.982272 (2020) doi:10.1101/2020.03.07.982272. Elnaggar, A. et al. ProtTrans: Toward Understanding the Language of Life Through Self-Supervised Learning. IEEE Trans. Pattern Anal. Mach. Intell. 44, 7112-7127 (2022). Madani, A. et al. Deep neural language modeling enables functional protein generation across families. bioRxiv 2021.07.18.452833 (2021) doi:10.1101/2021.07.18.452833. Bian, J., Dannappel, M., Wan, C. & Firestein, R. Transcriptional Regulation of Wnt/β-Catenin Pathway in Colorectal Cancer. Cells 9, (2020). Zhao, H. et al. Wnt signaling in colorectal cancer: pathogenic role and therapeutic target. Mol. Cancer 21, 1-34 (2022). Khalaf, A. M. et al. Role of Wnt/β-catenin signaling in hepatocellular carcinoma, pathogenesis, and clinical significance. J Hepatocell Carcinoma 5, 61-73 (2018). Sedan, Y., Marcu, O., Lyskov, S. & Schueler-Furman, O. Peptiderive server: derive peptide inhibitors from protein-protein interactions. Nucleic Acids Res. 44, W536-41 (2016). Radford, A. et al. Learning Transferable Visual Models From Natural Language Supervision. (2021) doi:10.48550/arXiv.2103.00020. Rao, R. et al. MSA Transformer.bioRxiv 2021.02.12.430858 (2021) doi:10.1101/2021.02.12.430858. Porras, P. et al. Towards a unified open access dataset of molecular interactions. Nat. Commun. 11, 1-12 (2020). Oughtred, R. et al. The BioGRID interaction database: 2019 update. Nucleic Acids Res. 47, D529-D541 (2018). Johnson, K. L. et al. Revealing protein-protein interactions at the transcriptome scale by sequencing. Mol. Cell 81, 4091-4103.e9 (2021). Palepu, K. et al. Design of Peptide-Based Protein Degraders via Contrastive Deep Learning. bioRxiv 2022.05.23.493169 (2022) doi:10.1101/2022.05.23.493169. Lin, Z. et al. Language models of protein sequences at the scale of evolution enable accurate structure prediction. bioRxiv 2022.07.20.500902 (2022) doi:10.1101/2022.07.20.500902. Mirdita, M. et al. ColabFold: making protein folding accessible to all. Nat. Methods 19, 679-682 (2022). Evans, R. et al. Protein complex prediction with AlphaFold-Multimer. bioRxiv 2021.10.04.463034 (2022) doi:10.1101/2021.10.04.463034. Huang, L., Guo, Z., Wang, F. & Fu, L. KRAS mutation: from undruggable to druggable in cancer. Signal Transduction and Targeted Therapy 6, 1-20 (2021). Graham, R. P. et al. DNAJB1-PRKACA is specific for fibrolamellar carcinoma. Mod. Pathol. 28, 822-829 (2015). Dong, X. C. PNPLA3—A Potential Therapeutic Target for Personalized Treatment of Chronic Liver Disease. Front. Med. 0, (2019). Hou, X., Zaks, T., Langer, R. & Dong, Y. Lipid nanoparticles for mRNA delivery. Nature Reviews Materials 6, 1078-1094 (2021). Tran, T. H. et al. KRAS interaction with RAF1 RAS-binding domain and cysteine-rich domain provides insights into RAS-mediated RAF activation. Nat. Commun. 12, 1-16 (2021). Zhan, T., Rindtorff, N. & Boutros, M. Wnt signaling in cancer. Oncogene 36, 1461-1473 (2016). Nusse, R. & Clevers, H. Wnt/β-Catenin Signaling, Disease, and Emerging Therapeutic Modalities. Cell 169, 985-999 (2017). Korinek, V. et al. Constitutive transcriptional activation by a beta-catenin-Tcf complex in APC-/-colon carcinoma. Science 275, 1784-1787 (1997). Ludwicki, M. B. et al. Broad-Spectrum Proteome Editing with an Engineered Bacterial Ubiquitin Ligase Mimic. ACS Cent Sci 5, 852-866 (2019). Cong, F., Zhang, J., Pao, W., Zhou, P. & Varmus, H. A protein knockdown strategy to study the function of beta-catenin in tumorigenesis. BMC Mol. Biol. 4, 10 (2003).

### SUMMARY

In one or more implementations of the subject matter described herein, systems and methods are provided for implementing a unified, sequence-based framework to design peptide-guided degraders without structural information, wherein such peptide-guided degraders can be used in diagnostic, analytic and therapeutic applications and compositions relating to the same.

In a particular implementation, the inventors have found that through the use of a pre-trained language models with protein interaction databases, it is possible to generate a unified, sequence-based framework to design peptide-guided degraders without structural information. As used throughout, such a sequence based system is referred to as a Structure-agnostic Language Transformer & Peptide Prioritization or SaLT&PepPr module. This SaLT&PepPr module efficiently down-selects peptides from known binding protein sequences for downstream screening.

In one or more implementations a system for generating a binding protein sequence configured to bind to a target protein sequence is provided. In a particular configuration, the system comprises a pre-trained prediction model, wherein the pre-trained prediction model includes a protein language model configured to output position data to a multi-layer perceptron classification neural network, wherein the perceptron classification neural network is configured to output values corresponding to the per-position probability of each amino acid sequence binding to the target sequence. The system is further configured to generate based on the output probability, a binding sequence configured to bind to the target sequence.

Such a system can further include one or more peptide synthesis devices configured to synthesize one or more peptide sequences generated by the pre-trained prediction model.

In a further arrangement, a peptide generation system is provided. The peptide generation system includes a search engine configured to search an interactome database; a predictive engine configured to generate a proposed binding sequence peptide based on the results of the search of the interactome database, and an output module configured to extract from the proposed binding sequence, subsequences predicted to have binding affinity for the target above a pre-determined threshold.

A method for predicting the similarity between target and peptide molecules is also provided, the method comprising: generating feature-rich embeddings for a plurality of target and peptide molecules using a pre-trained ESM-2 model; forming a matrix with rows corresponding to the target molecules and columns corresponding to the peptide molecules; predicting the cosine similarity between each pair of target and peptide molecules in the matrix using a trained model; calculating the average of the cross-entropy losses on the rows and columns of the matrix; and outputting the predicted cosine similarity and the average cross-entropy loss as a measure of the performance of the trained model.

In a particular arrangement, a method of generating binding peptide sequences to a target sequence is provided, the method comprising: receiving, using a processor configured by code executing therein, a data object corresponding to a protein target; searching, using the data object, a protein interaction database for at least one partner protein to the target protein; identifying at least one partner protein to the target protein; providing the at least one partner protein to a computational model configured to output a predicted protein sequence predicted to interact with the target sequence; and identifying at least one subsequence within the predicted protein sequence that meets a predetermined interaction threshold.

In one or more further implementations, a chimeric molecule is provided that includes one or more peptides generated using the SaLT&PepPr-derived sequence. In one arrangement such a chimeric molecule is used to effect post-translational modification of a target of interest. For example, the SaLT&PepPr-derived sequence is configured to link a target to one or more E3 ubiquitin ligase domains. Such a chimeric molecule can be used for targeted degradation of particular biological targets of interest.

In yet a further implementation, the SaLT&PepPr module can reliably identify candidates exhibiting robust intracellular degradation of diverse pathogenic targets in human cells, including those with minimal structural information.

In yet a further arrangement, a peptide-guided degraders are provided where the peptide was generated using the SaLT&PepPr module, and such peptide-guided degrader has negligible off-target effects via whole-cell proteomics. Such peptide-guided degraders able to demonstrate the degradation of endogenous-catenin and subsequent downregulation of Wnt signaling in cellular models of colorectal cancer and thus have utility as therapeutics for a number of potential ailments.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a block diagram detailing the arrangement of elements of the system described herein in accordance with one embodiment of the invention.

FIG. 2 is a schematic showing the relationship of various modules of the system described.

FIG. 3 is a flow diagram detailing the process of generating a new peptide sequence.

FIG. 4 a chart detailing the percentage of proteome with documented protein protein interactions.

FIG. 5 is a flow diagram detailing the data flow within one embodiment of the described model.

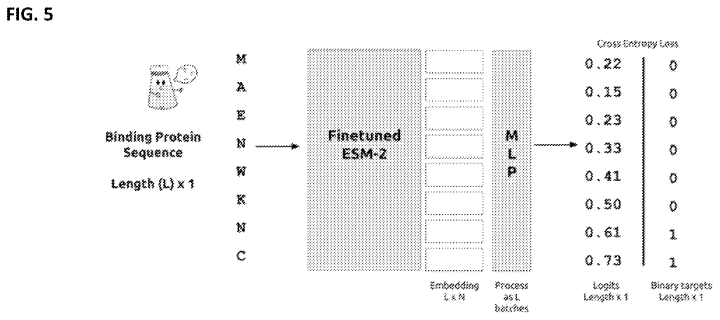

FIG. 6 is a table detailing performance of the present approach relative.

FIG. 7 is a table benchmarking the model against different protein prediction approaches.

FIG. 8 illustrates a comparison of the output of a model of the present description compared with alternative approaches.

FIG. 9 illustrates a comparison of the output of a model of the present description compared with alternative approaches.

FIG. 10 details a flow diagram of the steps of generating a new peptide sequence based on a target input.

FIGS. 11A-11D details characterization of derived uAbs for targeted modulation.

FIGS. 12A-12D details characterization of derived uAbs for targeted modulation.

### DETAILED DESCRIPTION

By way of overview and introduction, the subject matter of the present application concerns systems and methods of generating peptides that can bind to an identified target. In particular, the present description provides for a system, method and approach that allows for generating peptides that does not require the use of structural information and is based on sequence data alone.

Inspired by the programmability of RNA-guided genome editing the described systems, methods and computer implemented approaches utilize one or more pre-trained protein language models to design short “guide” peptide sequences that bind to user identified or computationally identified target proteins.

By training on in silico-derived predictions of binding affinity on protein interfaces, it is possible to isolate peptide fragments that have significant contribution to the overall binding energy of a target protein. Furthermore, by leveraging predicted or experimentally-validated binding proteins to specified target proteins as starting scaffolds for splicing, a Structure-agnostic Language Transformer & Peptide Prioritization (SaLT&PepPr) module can be utilized to generate peptides that have a substantial likelihood of binding to the target protein.

In one or more particular arrangements, these designed peptide sequences are used as a component of a chimeric molecule that are designed to induce post-translational modifications. For instance, and in no way limiting, the designed peptide sequences when fused to modular E3 ubiquitin ligase domains are configured to induce degradation of the target. However, other post-translational modifications can be used with the described computationally designed peptide sequences.

For example, in one or more implementations, a system for generating a binding protein sequence configured to bind to a target protein sequence is provided. In a particular configuration, the system comprises a pre-trained prediction model, wherein the pre-trained prediction model includes a protein language model configured to output position data to a multi-layer perceptron classification neural network, wherein the perceptron classification neural network is configured to output values corresponding to the per-position probability of each amino acid sequence binding to the target sequence. The system is further configured to generate, based on the output probability, a binding sequence configured to bind to the target sequence.

The described system of sequence based binding prediction, SaLT&PepPr, reliably and efficiently generates peptides. When these peptides are integrated within a uAb construct, the resulting construct can induce robust post translational modifications. For example, and in no way limiting, such approaches can be used to induce degradation of diverse pathogenic targets in human cells. For example, the described approach can be used to develop degraders to β-catenin, whose cytosolic variant can cause aberrant Wnt signaling, leading to numerous forms of cancer, including colorectal and hepatocellular carcinomas. In particular, SaLT&PepPr-designed uAbs bind with high affinity, induce degradation of endogenous-catenin, and subsequently downregulate Wnt signaling in cellular models of colorectal cancer, thus motivating clinical translation of such a uAb platform.

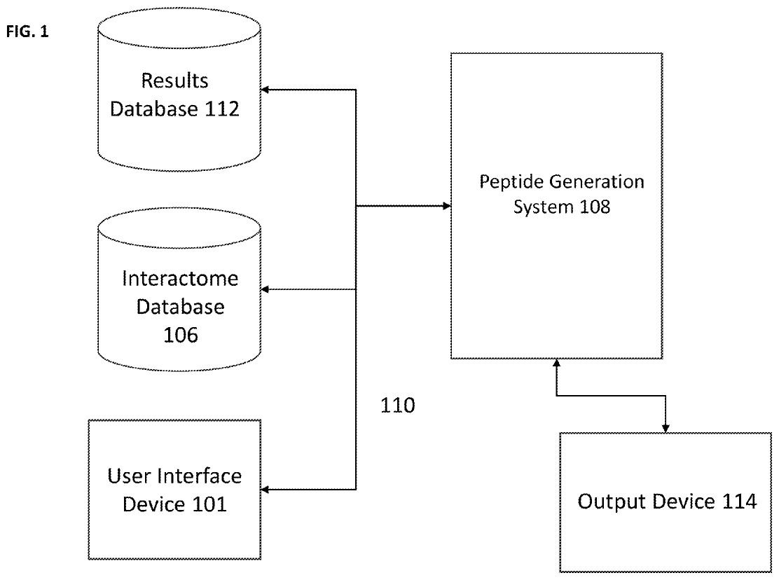

Turning now to FIG. 1, the described system includes a user interface device 101, an interactome database 106, a peptide generation system 108 and one or more output devices 114.

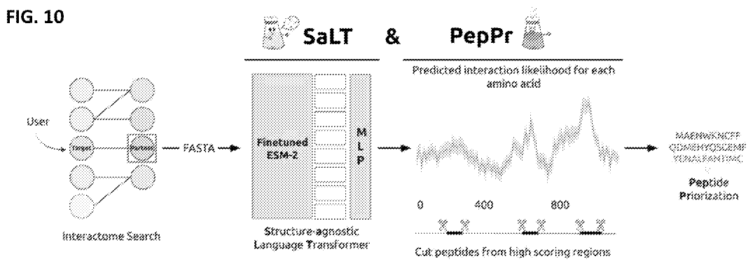

As shown in FIG. 1 and FIG. 10, protein target selections made by a user of a user interface device 101 are provided to a processor 108. In one particular implementation, the user interface device 101 is a standard computing device, such as a desktop or portable computing device. However, in particular arrangements, the user interface device 101 is custom computing platform specifically designed to carry out the tasks described herein.

As shown in FIG. 1 user interface device 101 is configured to transmit one or more protein selections to peptide processing platform, such as processing platform 108. In one or more configurations, the user interface device 101 is equipped or configured with network interfaces or protocols usable to communicate over a network, such as the internet. In this configuration, selection made by the user can be from a stored local collection of proteins, a public database of proteins, or another source of protein data.

Alternatively, user interface device 101 is connected to one or more computers or processors, such as processing platform 108, using standard interfaces such as USB, FIREWIRE, Wi-Fi, Bluetooth, and other wired or wireless communication technologies suitable for the transmission protein data.

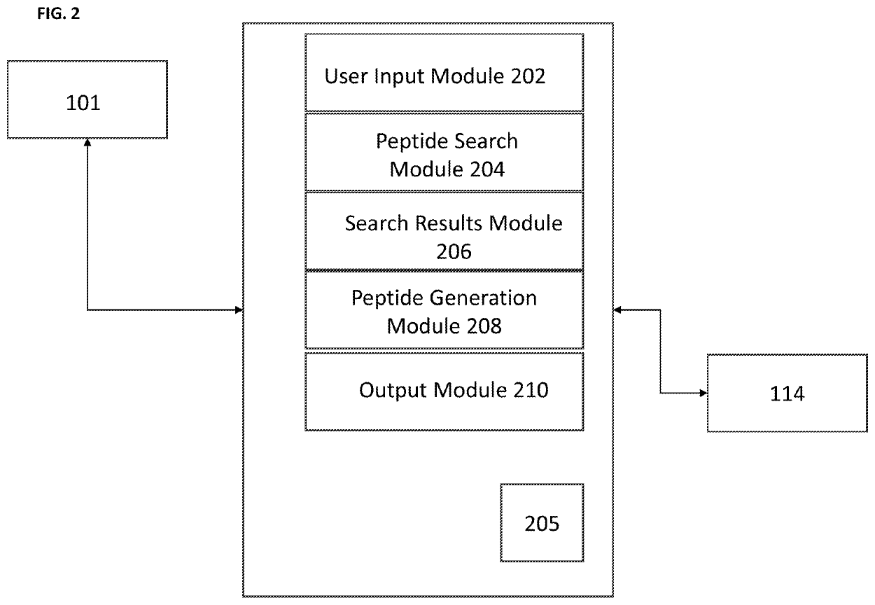

The protein data generated or transmitted to the one or more processing platform(s) 108 is then evaluated as a function of one or more hardware or software modules. As used herein, the term “module” refers, generally, to one or more discrete components that contribute to the effectiveness of the presently described systems, methods and approaches. Modules can include software elements, including but not limited to functions, algorithms, classes and the like. In one arrangement, the software modules are stored as software in memory 205 of processing platform 108, as shown in FIG. 2.

Modules can, in some implementations, include discrete or specific hardware elements. In one implementation, user interface device 101 is located within the same device as the processing platform 108. For example, each of the user interface 101 and the processing platform 108 are software modules executed by one or more processors of a server, computing cluster or other data processing and execution platform. However, in another implementation, processing platform 108 is remote or separate from the user interface device 101 and communicates over one or more communication linkages.

In one configuration, processing platform 108 is configured through one or more software modules to generate, calculate, process, output or otherwise manipulate the data provided by the user interface device 101.

In one implementation, processing platform 108 is a commercially available computing device. For example, processing platform 108 may be a collection of computers, servers, processors, cloud-based computing elements, micro-computing elements, computer-on-chip(s), home entertainment consoles, media players, set-top boxes, prototyping devices or “hobby” computing elements.

[Furthermore, processing platform 108 can comprise a single processor, multiple discrete processors, a multi-core processor, or other type of processor(s) known to those of skill in the art, depending on the particular embodiment. In a particular example, processing platform 108 executes software code on the hardware of a custom or commercially available cellphone, smartphone, notebook, workstation or desktop computer configured to receive data or measurements captured by the sample color sensors 106 either directly, or through a communication linkage.

Processing platform 108 is configured to execute a commercially available or custom operating system, e.g., Microsoft WINDOWS, Apple OSX, UNIX or Linux based operating system in order to carry out instructions or code.

In one or more implementations, processing platform 108 is further configured to access various peripheral devices and network interfaces. For instance, processing platform 108 is configured to communicate over the internet with one or more remote servers, computers, peripherals or other hardware using standard or custom communication protocols and settings (e.g., TCP/IP, etc.).

Processing platform 108 may include one or more memory storage devices (memories). The memory is a persistent or non-persistent storage device (such as an IC memory element) that is operative to store the operating system in addition to one or more software modules. In accordance with one or more embodiments, the memory comprises one or more volatile and non-volatile memories, such as Read Only Memory (“ROM”), Random Access Memory (“RAM”), Electrically Erasable Programmable Read-Only Memory (“EEPROM”), Phase Change Memory (“PCM”), Single In-line Memory (“SIMM”), Dual In-line Memory (“DIMM”) or other memory types. Such memories can be fixed or removable, as is known to those of ordinary skill in the art, such as through the use of removable media cards or modules. In one or more embodiments, the memory of processing platform 108 provides for the storage of application program and data files. One or more memories provide program code that processing platform 108 reads and executes upon receipt of a start, or initiation signal.

The computer memories may also comprise secondary computer memory, such as magnetic or optical disk drives or flash memory, that provide long term storage of data in a manner similar to a persistent memory device. In one or more embodiments, the memory of processing platform 108 provides for storage of an application program and data files when needed.

The processing platform 108 is configured to store data either locally in one or more memory devices. Alternatively, processing platform 108 is configured to store data, such as data or processing results, in a local or remotely accessible database 112. The physical structure of database 112 may be embodied as solid-state memory (e.g., ROM), hard disk drive systems, RAID, disk arrays, storage area networks (“SAN”), network attached storage (“NAS”) and/or any other suitable system for storing computer data. In addition, database 112 may comprise caches, including database caches and/or web caches. Programmatically, database 112 may comprise flat-file data store, a relational database, an object-oriented database, a hybrid relational-object database, a key-value data store such as HADOOP or MONGODB, in addition to other systems for the structure and retrieval of data that are well known to those of skill in the art. Database 112 includes the necessary hardware and software to enable the processing platform 108 to retrieve and store data within database 112.

In one implementation, each element provided in FIG. 1 is configured to communicate with one another through one or more direct connections, such as though a common bus. Alternatively, each element is configured to communicate with the others through network connections or interfaces, such as a local area network LAN or data cable connection. In an alternative implementation, user interface device 101, processing platform 108, and database 112 are each connected to a network 110, such as the internet, and are configured to communicate and exchange data using commonly known and understood communication protocols.

In a particular implementation, processing platform 108 is a computer, workstation, thin client or portable computing device such as an Apple ipad/iPhone® or Android® device or other commercially available mobile electronic device configured to receive and output data to or from database 112 or 106.

In one arrangement, processing platform 108 communicates with an output device 114 to transmit, generate, displaying or exchange data. In one arrangement, the output device 114 and processing platform 108 are incorporated into a single form factor, such as a computing device that is connected to one or more protein synthesis devices or systems. For example, such devices or systems could be workbench, or bench-top protein synthesis devices. Here, such an integrated system includes one or more computers or other data processing devices, tools, devices or reaction or synthesis elements necessary to synthesize peptide sequences.

Those possessing an ordinary level of skill in the requisite art will appreciate that additional features, such as power supplies, power sources, power management circuitry, control interfaces, relays, adaptors, and/or other elements used to supply power and interconnect electronic components and control activations are appreciated and understood to be incorporated.

As shown in FIGS. 1, 2 and 10, a user submits a protein target to the system. This protein target is used to search an interactome database (such as database 106). For example, a processor of the user interface device 101 is configured with one or more connections to an interactome database 106. Here, the user is able to search the contents of the interactome database 106 for a protein target of interest. Upon selection of a target or targets of interest, a processor of the user interface device 101 is configured to send the user selection to the processing platform 108. Here, the processing platform 108 is configured by a user input module 202 to receive the user protein (target) selection. For example, the user input module 202 configures one or more processors of the processing platform 108 to receive a data file, object, stream or link generated by the user input device 101.

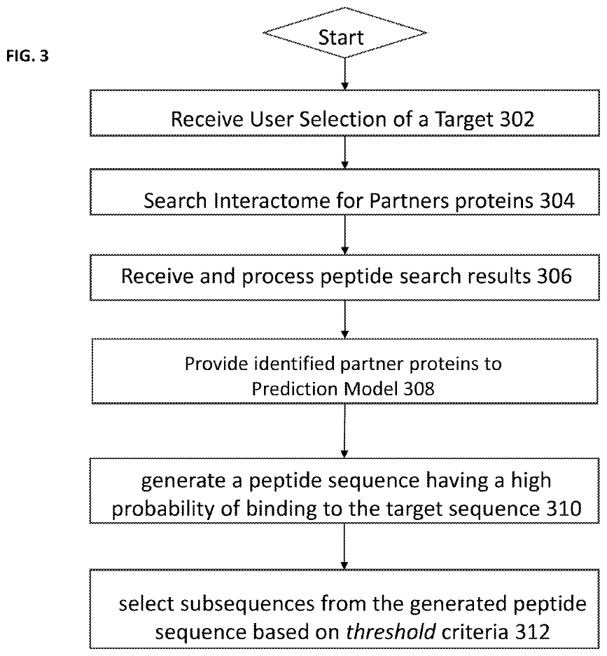

As shown in step 302 of FIG. 3, the user's selection is passed to the processing platform 108 for further manipulation. In one arrangement, the processing platform 108 transmits the user's selection directly to the interactome database 106 in order to identify one or more partner proteins. In one particular implementation, the processing platform 108 alters or otherwise manipulates the user's selection prior providing the data to the interactome database 106.

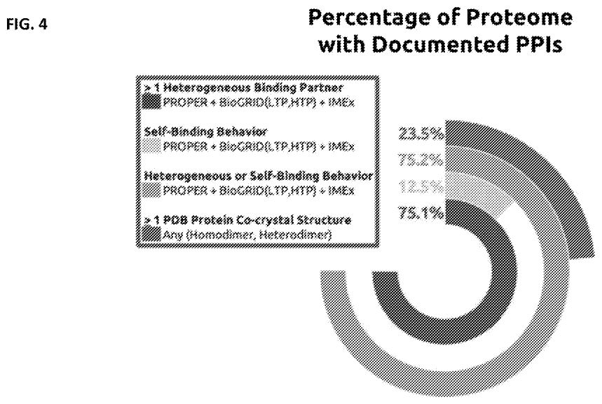

As noted, and with reference to FIG. 4, there exists multiple databases of protein interactions. However, not all protein databases are suitable for use in the systems and methods described herein.

In order to create a comprehensive dataset of computationally-derived presumptive peptides, in one arrangement the database, such as the interactome database, is generated by applying PeptiDerive to all PDB co-crystal complexes of high resolution (<2.5 Å), generating a total of ˜100 million peptide-target pairs with associated binding interface scores. Such a database represents a comprehensive collection of peptide-protein pairs and can thus serve as a standardized training set for interface modeling.

It will be further appreciated that the percentage of the human proteome with at least one binding partner can, in one arrangement, be estimated by screening three databases: IMEx (https://www.imexconsortium.org/), BioGRID (https://thebiogrid.org/), or PROPER (https://genemo.ucsd.edu/proper/)6-8.

In one arrangement, only those databases that explicitly provide experimental evidence of physical binding are used to create the interactome database 106. For example, in one arrangement, StringDB another protein interaction database which does not guarantee physical interaction is excluded. However, in one or more further arrangement of the systems, methods and approaches described, any data source of protein interaction can be included.

In one particular arrangement, the gene symbols corresponding to each human protein are accessed from a dataset. For example, gene symbols are downloaded from UniProt (20601 total). For each database, pandas was used to scan for symbols and compile lists of proteins involved in at least one PPI. Screening was performed separately for heterogeneous interactions and homogeneous (self-binding) interactions. To account for varying curation standards, the entire process is, in one arrangement, repeated twice with different sets of filters. The most stringent or least inclusive filtering included PROPER entries with p<0.01, all IMEx entries, and BioGRID entries justified by low throughput (LTP) physical evidence. The least stringent or most inclusive filtering included PROPER entries with p<0.05, all IMEx entries, and BioGRID entries justified by either LTP or high throughput (HTP) physical evidence.

To quantify the availability of structural data on PPIs, the protein database (PDB) was scanned for co-crystal complexes of two human proteins. Complexes were divided into two categories: heteromeric and homomeric. The PDB provides an Entry ID for each co-crystal and FASTA sequences for its two constituent proteins.

Because species indications and constituent Entry IDs were not directly available, determining the co-crystal composition can include a multistep process: (i) mapping co-crystal Entry IDs to organisms and filtering for human-human interactions only (reference: source.idx from the PDB archive, https://ftp.wwpdb.org/pub/pdb/derived_data/index/) (ii) mapping the constituent proteins in each cocrystal to Entry IDs based on their FASTA sequences (reference: pdb_seqres.txt from the PDB archive, https:/ftp. wwpdb.org/pub/pdb/derived_data/) (iii) mapping Entry IDs to UniProt KB identifiers and UniProt gene symbols (references: SIFTS database pdb_chain_uniprot. csv, https:/www.ebi.ac.uk/pdbe/docs/sifts/quick.html, UniProt Retrieve/ID Mapping tool) (iv) comparing the list of PDB-derived UniProt gene symbols to the full human genome. The final result represents the total number of human proteins involved in at least one co-crystal in PDB.

For example, in one implementation the PDB-derived dataset is generated by mining the RCSB PDB for verified, high-resolution PPI structures. For example, the process of extracting useful data begins by retrieving every interaction of every assembly of every cocrystal in the PDB. Then the interactions are filtered for uniqueness (a unique interaction was one with a unique pair of partners, or with significantly different (>100 Å2) buried surface area for the same pair of partners). In one particular example, this filtration step yielded 420,000 PPIs. Next, all interaction structures with an amino acid sequence length greater than 50 and less than 1023 (for computational training speed) were processed with Rosetta PeptiDerive. However, it will be appreciated amino acid sequences between 11 and 35,000 (TAL to Titin) can be used in the creation of the necessary datasets. Next, a list of derived peptides and their associated Rosetta energy scores (REUs) are extracted. In this implementation, the lower scores indicate higher predicted stability. Next, entries are filtered for those with lower than −1000 REU. Then, the REU scores for 10-mer peptides at each position were averaged to estimate a per amino acid position energy score. Next, the per position energy score is averaged between matching derived protein sequences, so that the dataset does not include redundant entries. A threshold value for energy score is then set, for example at −1. A binary classification task is established, with less than −1 energy being a protein binding amino acid and energy greater than −1 being a non-binding amino acid.

Returning to FIG. 3, the interactome database 106 is searched using the user's input generated in step 302, as further shown in step 304. In one arrangement, the processing platform 108 is configured by a peptide search module 204 to access the interactome database 106 and search the interactome database using one or more search criteria based on the user's selection as shown in step 304. For example, the peptide search module 204 causes the processing platform 108 to access an interactome database, such as database 106. Here, the peptide search module 204 further configures the processing platform to submit the target protein obtained in step 302 to a search program or algorithm.

As shown in step 306, the results of the search conducted in step 304 are provided back to the processing platform 108 as shown in step 306. For example, the processing platform 108 is configured by the search results module 206 to receive the search results from the interactome database 106. In one particular arrangement, the search results module 206 configures one or more processors of the processing platform 108 to generate a file or data object containing the results of the search of the interactome database. In a particular further arrangement, the search results module 206 configures one or more processors of the processing platform 108 to convert the returned partner sequence(s) into FASTA format, as shown in step 306.

Next, as shown in step 308, the search results are provided to the predictive model. In one arrangement the FASTA converted forms of the search results are evaluated by the processing platform 108. However, in alternative configurations, the direct results of the search conducted in 306 are directly evaluated by the processing platform 108. For example, the peptide generation module causes the processing platform 108 to access a computational model configured to receive input data in the form of a FASTA sequence, and generate an output value in the form of a nucleotide sequence or amino acid or sequences.

In one or more arrangements, the computational model developed is configured to generate putative partner proteins for a given target. This model utilizes the data derived from the curated PPI databases constructed as described herein.

For example, in or more implementations, the computational model implemented or accessed by the peptide generation module 208 is a machine learning model created to generate binding partners for target sequences using the data obtained in from the custom PPI databases. In one arrangement, the machine learning model is a large language model. In an alternative configuration, the machine learning model is a neural network. For example, a pre trained neural network is used to evaluate the data in the PPI database and generate binding partners for a target protein. In another configuration, the machine learning model is a protein language model.

In one arrangement, the machine learning model is a multi-million parameter protein language model. For example, the machine learning model is a 650-million parameter ESM-2 model. It will be understood that the ESM-2 model is a protein language model from Meta AI. However, it will be understood and appreciated that other protein language models are applicable. For example, any protein language model which enables featurization of protein interactions without the need for multiple sequence alignment (MSA) generation can be used. While the lack of MSA features, a notoriously costly derivation procedure, represents an improvement in the art of machine learning model, it will be appreciated that any machine learning model, including those that use MSA features, can be used in the foregoing systems, methods and approaches described herein.

By way of a more detailed overview, a person of ordinary skill in the requisite art would appreciate that ESM-2 is a transformer-based model uses a self-attention mechanism to learn the long-range dependencies between amino acids in a protein sequence. The self-attention mechanism allows the model to learn how different parts of a protein sequence interact with each other, which is essential for understanding protein structure and function. Thus, any model that incorporates this or a similar feature is suitable for the approaches described herein.

Furthermore, ESM-2 consists of a stack of encoder layers, each of which contains a self-attention layer and a feed-forward layer. The encoder layers are responsible for extracting the underlying features from the input protein sequence. The output of the encoder is fed into a decoder layer, which is responsible for generating the embedding for the input protein sequence. In one arrangement, the decoder layer is a transformer layer with a different architecture than the encoder layers. For example, ESM-2 is trained using a masked language modeling objective. As such, the ESM-2 model is trained to predict masked amino acids in the input protein sequence. This objective allows the model to learn the relationships between amino acids in a protein sequence and to encode a wide range of biological information in its embeddings.

The machine learning model provided is, in one or more implementations, be fine-tuned on a dataset of labeled examples. For example, to fine-tune ESM-2 for protein structure prediction, the model would be trained on a dataset of protein sequences stored in the interactome database or other protein database.

Here, the present approaches further alter the ESM-2 model by fine tuning the final three layers of the ESM-2 model. These final layers are fine-tuned using the data from the protein interaction (PPI) dataset, interactome database or other protein database.

In addition, the ESM-2 model is then paired with a classification head. In one particular configuration, the classification head is a neural network configured to receive the output of the fine-tuned ESM-2 model. In a further arrangement, the neural network is a perceptron classification head. For example, the classification head is a multilayer perceptron (MLP) classification head that is paired with the fine-tuned ESM-2 model. Here, the MLP is a neural network that can is configured to perform classification tasks. An MLP consists of fully connected neurons with a nonlinear activation function, organized in at least three layers. Here, the MLP takes the embeddings from ESM-2 as input and outputs the predicted class for each amino acid in the sequence. For instance, the MLP is trained to classify the per amino acid interacting positions.

By way of a more detailed example, the final three layers of ESM-2 650 M are fine tuned together with a four layer fully connected neural network classification head which processes each position output of ESM-2 to predict a per position probability. Here, the model is configured such that each protein is passed to the model with the per amino acid binary class as the target for cross entropy loss: −(ylog(p)+(1−y)log(1−p)) where y is the binary class label (0=nonbinding and 1=binding) and p is the predicted probability of the amino acid belonging to a binding site.

More specifically, the computational model is configured to learn how to predict how similar two different sequences are to each other. To do this, the computational model is trained on a dataset of sequences that have been labeled as either similar or not similar. The model first generates a representation of each sequence, called an embedding. An embedding is a vector of numbers that represents the information about the sequence. The model then calculates the cosine similarity between the embeddings of each pair of sequences. Cosine similarity is a measure of how similar two vectors are. The higher the cosine similarity, the more similar the two sequences are. The model is then trained to predict the cosine similarity between each pair of sequences in the dataset. The model's predictions are compared to the actual cosine similarities, and the model is updated to improve its predictions. The average of the cross-entropy losses on the rows and columns of the matrix is used to measure how well the model is performing. Cross-entropy loss is a measure of how different two probability distributions are. The lower the cross-entropy loss, the better the model is performing.

In a particular implementation, the ESM-2 model and its MLP header is implemented using PyTorch and trained until validation loss began to increase. When using the PPBS dataset, the weighting method used in Tubiana, et al., was adopted by multiplying the loss by the specified weight for consistency with ScanNet.

It will be appreciated that in one or more implementations, the machine learning module accessed by the peptide generation module 208 is a pre-trained model. However, in one or more implementations a peptide generation module implements one or more submodules to train a computational model prior to outputting peptide sequences.

FIn one particular arrangement, training, validation and testing sets were created with 26,423 train, 3487 validation, and 3817 test sequences, with no entries across different sets belonging to the same cluster. Such an approach ensures that validation and test metrics do not reflect memorization of the properties of homologous protein sequences. Proteins which were clustered by MMseqs to partner proteins selected for in vitro testing were also moved to the test set. For benchmarking, the Dockground-based PPBS dataset used in ScanNet was utilized.

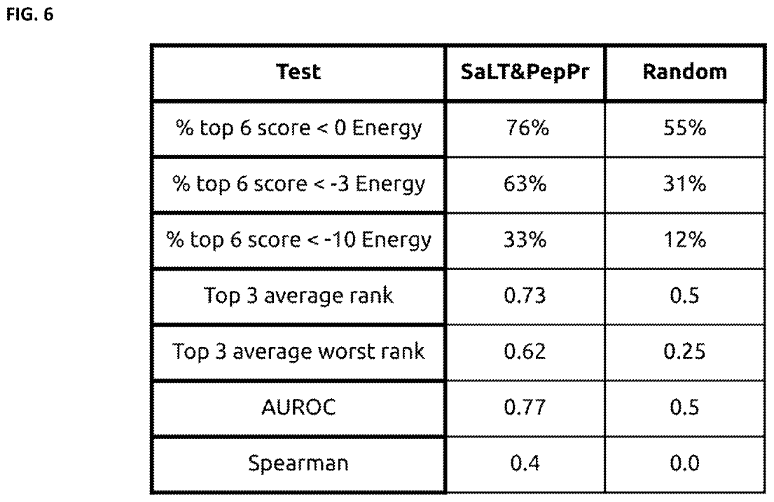

The developed model, referred to herein as a Structure-agnostic Language Transformer & Peptide Prioritization (SaLT&PepPr) module, is configured to utilize the entire partner sequence. As such, the SaLT&PepPr model described herein outperforms Cut&CLIP in ranking peptides from the same protein, with a final Spearman correlation of 0.4 compared to <0.05 for Cut&CLIP on the test set (see FIG. 6). Thus, the described model represents an improvement in the technological field of protein-protein interaction modeling and other computational approaches to evaluating proteins.

It will be appreciated and understood that the SaLT&PepPr inference model and module described herein is, in one or more implementations, integrated with existing gold-standard, experimentally-validated PPI datasets, including IMEx, BioGRID, and PROPER. Such datasets cover over 75% of all target proteins in the human proteome (as opposed to <25% with an existing co-crystal). The SaLT&PepPr module can, in one or more further implementations, be further extended by utilizing multimeric targets as their own scaffolds for peptide derivation For example, see FIG. 6. Persons of ordinary skill in the relevant art would appreciate that multimeric targets are proteins that are made up of two or more subunits. These subunits can be identical or different. Peptide scaffolds are proteins that can be used to display peptides on their surface. Thus, the SaLT&PepPr module can be used to design peptides that can bind to the subunits of multimeric targets. This would allow the peptides to have a stronger binding affinity to the targets than if they were only binding to one subunit. Furthermore, the SaLT&PepPr module is configured to derive peptides from the amino acid sequence of the multimeric targets. Such an approach allows for the design of peptides to that specifically bind to the multimeric targets.

As shown in step 310, the predictive model evaluates the search result data. For instance, the processing platform 108 is configured by a peptide generation module 208 that provides an instance of the predictive model as described herein. Here, the processing platform 108 is configured by the peptide generation module 308 to generate a peptide sequence that is predicted to bind to the target sequence identified by the user in step 202.

Once the peptide sequence has been generated, it can be outputted as shown in step 312. For example, the output module 310 configures one or more processors of the processing platform 108 to evaluate the generated sequence and select subsequences for further use or analysis. For example, the output module 310 is configured to evaluate the output of the predictive model, and provides for each amino acid, the likelihood of an interaction with the target sequence.

The output module 310 is configured, in one arrangement, to identify subsections of the generated sequence based on a likelihood threshold. For example, where the predicted interaction for a grouping of amino acids in the generated sequence is above a pre-determined threshold (such as above 20, 30, 40, 50, 60, 70, 80, 90, 95 probability of an interaction), the output module selects those grouping and outputs those groupings, or subsequences, for further use and evaluation. For example, in one or more arrangements, both the target information and the predicted interaction sequences are stored in the results database 112 for further use and access. Additionally, the computational model (SaLT&PepPr) is configured to generate peptides that can be sampled across the partner sequence to both maximize breadth of selection and incorporate prior knowledge of known binding domains. Specifically, the SaLT&PepPr module predicts the probability of each amino acid in the partner protein sequence being an interaction site.

As part of this prediction process, continuous peptides are “cut” from the full partner sequence to select the segment with the highest average predicted score. In one arrangement, in order to sample different regions across a partner protein, a greedy sampling approach is used to take peptides of a user-specified length with highest predicted probability of binding, as shown in FIG. 10.

In will be appreciated that as the SaLT&PepPr computational model is a purely sequence-based model, the total inference time for a single target protein takes about one minute on a standard machine with 2 CPU cores, 8 GB of RAM, and no GPU. Thus, the SaLT&PepPr module represents a non-routine, non-conventional improvement in the art of protein interaction modeling. For example, when compared the SaLT&PepPr, a functionally equivalent method of employing the openly accessible ColabFold software plus a PeptiDerive step for an interacting sequence pair requires over one hour of compute time, and cannot reliably operate on large, multimeric complexes due to hardware limitations.

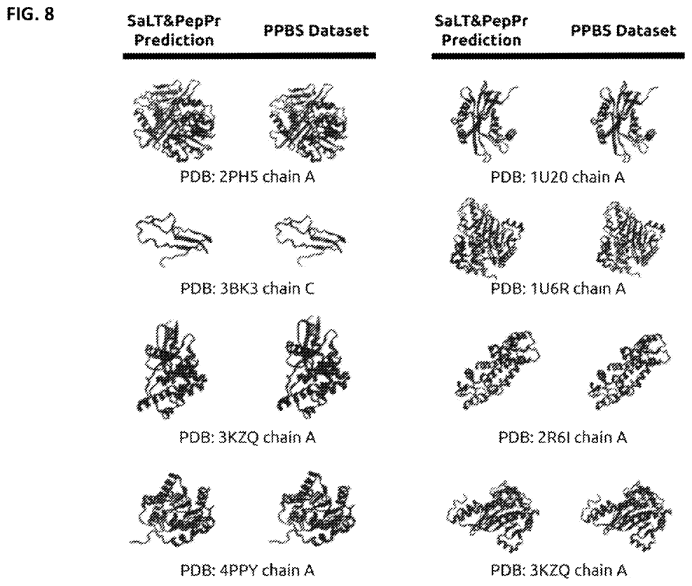

For example, as shown is FIG. 8, details a comparison between the predicted SaLT&PepPr scores and experimentally-annotated PPBS binding sites on different protein structures in the PPBS dataset. Red indicates high binding probability amino acids, with blue as low binding probability, normalized for each protein chain.

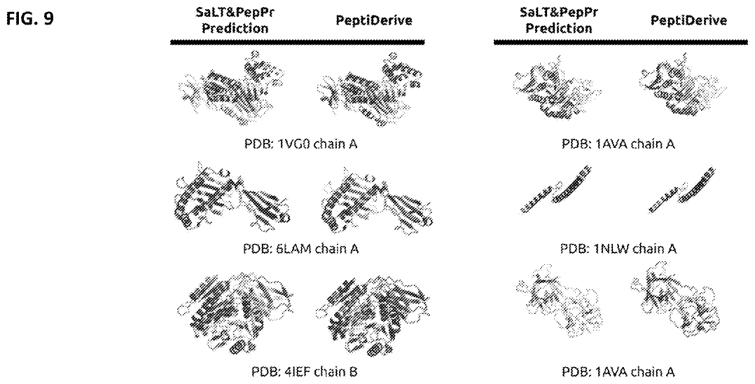

Likewise, FIG. 9 illustrates representative examples of model inference versus calculated PeptiDerive energy landscapes from specific PDB co-crystal entries. Red indicates high binding probability, with white as lower and blue as low, and gray indicates amino acids which are discarded because of being invalid for PeptiDerive. Note that PeptiDerive scores visualized only reflect binding sites captured in the specific PDB entry.

Overall, the computational model described herein exhibits robust model performance across multiple target proteins, especially those with known binding domains. When trained and tested on the PDB-derived dataset, SaLT&PepPr achieved a test set area under the ROC curve (AUROC) of 0.77, as shown in FIG. 6. Alternatively, when keeping ESM-2 weights frozen, the test AUROC was 0.7, demonstrating the benefit of fine-tuning the final layers of the original model. This approach, which utilizes the sequence of the binding partner, has a Spearman correlation to PeptiDerive energy scores of 0.4 on the test set with sequence homology <25%.

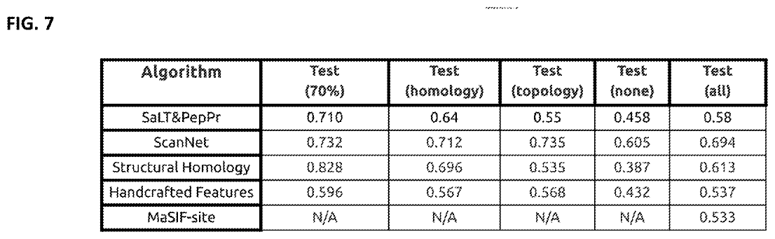

Computational models were also trained and tested on the ScanNet PPBS dataset to compare the described computational model to baseline and state-of-the-art models which require tertiary structure and/or multiple sequence alignments (MSAs) to identify protein interacting residues. Despite not using structure as input, the described computational models achieved competitive performance compared to structure-based benchmarks, and decreased performance compared to ScanNet, as shown in FIG. 7. Specifically, on the “Test none” split which reflects most distant proteins, SaLT&PepPr exhibited superior performance to baseline methods based on structural homology and handcrafted feature selection, suggesting strong generalization to non-homologous proteins from different families. Finally, we visualized SaLT&PepPr predictions on partner proteins with available crystal structures from the PDB, highlighting the model's capability to identify concise interacting interfaces, both in isolated structures of single binding proteins, as shown in FIG. 8, and within co-crystals, as shown in FIG. 9.

### Experimental Testing

Recently, based on the seminal work of Portnoff et al.1, our group reprogrammed the specificity of a modular human E3 ubiquitin ligase called CHIP (carboxyl-terminus of Hsc70-interacting protein) by replacing its natural substrate-binding domain, TPR, with designer “guide” peptides to generate minimal and programmable uAb architectures. To demonstrate that guide peptides derived from known, selected interacting partners can function as robust guide peptides, we first focused on designing uAbs to β-catenin, as aberrant Wnt/βcatenin signaling is widely implicated in numerous cancers, including colorectal, hepatocellular, lung, and pancreatic.

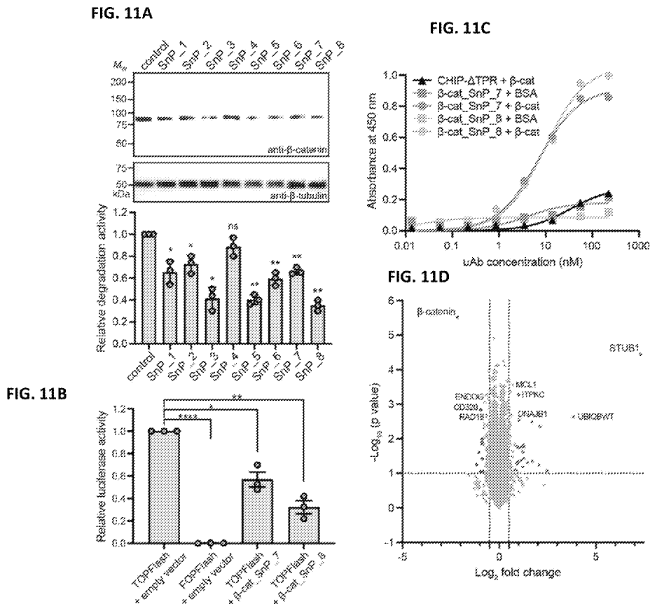

Specifically, mutated β-catenin accumulates in the cytosol of affected cells, while wild-type β-catenin binds to the transmembrane protein, E-cadherin20. Thus, to enable degradation of endogenous, cytosolic β-catenin, we leveraged its known sequence interaction with E-cadherin to select guide peptides from the Ecadherin/β-catenin binding interface for subsequent uAb generation, and scored them with SaLT&PepPr. We then transfected DLD1 colon cancer cells, which express wild-type β-catenin at abnormally high levels, with our uAb constructs (SnP_1 to SnP_8). Immunoblots of the cytosolic fractions revealed that all but one uAb promoted statistically significant β-catenin degradation relative to non-transfected DLD1 control cells, with several (SnP_3, SnP_5, SnP_8) degrading >60% of the cytosolic β-catenin pool (FIG. 11a). For example, a Degradation of endogenous β-catenin in cytosolic fraction of DLD1 cells analyzed via immunoblotting with anti-β-catenin and anti-β-tubulin antibodies. Blots are representative of independent transfection replicates (n=3). Relative degradation activity was determined by densitometry analysis of anti-β-catenin immunoblot. b TOPFlash luciferase reporter assay of βcatenin/TCF transcriptional activity. FOPFlash reporter served as negative control. c β-catenin binding activity determined by ELISA with immobilized βcatenin (β-cat). Binding to bovine serum albumin (BSA) served as negative control. d Nano LC-MS/MS analysis of total proteins collected from HEK293T cells co-transfected with plasmids encoding SnP_8 uAb and β-catenin-sfGFP. Data were log2-normalized and fold-change and p-value (unpaired, two-tailed t-test) were performed to generate volcano plot of differentially abundant proteins. STUB1 denotes overexpressed CHIPΔTPR domain of SnP_8 uAb. Data in a-c are the average of independent transfection replicates (n=3)±SD. For individual samples, statistical significance was determined by twotailed Student's t test.

Using TOPFlash22, a luciferase reporter that serves as a reliable readout of β-catenin-dependent transcriptional activity, we observed that the strong SnP_8 degrader dramatically decreased the transcriptional response to β-catenin relative to empty vector control cells (FIG. 11b). For comparison, the SnP_7 degrader induced a more modest inhibitory effect on β-catenin signaling, in line with its intermediate degradation activity. We confirmed that peptide-guided uAbs promoted target degradation through specific, peptide-mediated binding of βcatenin as demonstrated by quantitative ELISA (FIG. 11c). Specifically, purified versions of SnP_7 and SnP_8 uAbs exhibited strong affinity to immobilized β-catenin with virtually no binding to the immobilized bovine serum albumin (BSA) control. The strong β-catenin binding exhibited by SnP_7 and SnP_8 was attributable to the SaLT&PepPr peptides as evidenced by the lack of binding for the CHIPΔTPR ubiquitination domain alone. We note that the relatively high binding activity of these uAbs for βcatenin was in line with the binding affinity measured for other uAbs1,23. Given the similar binding activity yet different levels of β-catenin silencing, other factors such as proximity/orientation upon binding must also contribute to the efficacy of peptide guided uAbs. Finally, to test the off-targeting propensity of our peptide guided uAbs, one dimensional liquid chromatography-tandem mass spectrometry (1D-LC-MS/MS) analysis was performed on total proteins harvested from cells overexpressing β-catenin, with or without treatment with the uAb candidates, with ˜6700 proteins were quantified. Our analysis demonstrated the expected increase in uAb-associated proteins, including tryptic peptides assigned to the human CHIP protein (STUB1), and a corresponding decrease in β-catenin abundance between the control and treated samples for both tested uAbs (FIG. 11d). In contrast, there were no significant changes in the abundance of other proteins as a function of uAb expression, confirming that there were no statistically significant off-target effects associated with uAb expression or degradation.

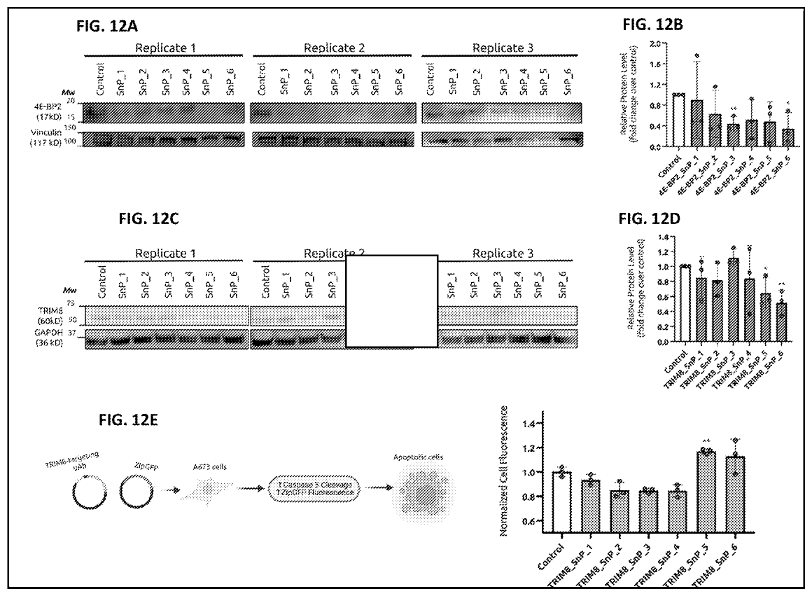

Experimental validation of SaLT&PepPr interface prediction for endogenous target degradation. Having established the ability to use interacting partners as effective scaffolds for guide peptide generation, we sought to test SaLT&PepPr's ability to prioritize effective guide peptides in a data-driven manner. To do this, we first chose eukaryotic initiation factor 4E binding protein 2 (4E-BP2), a relatively small and disordered protein involved in eukaryotic translation initiation that has also been implicated in cancer24,25. 4E-BP2 has a single known specific interactor: eukaryotic initiation factor 4E (elF4E)26. Using the eIF4E as input into SaLT&PepPr, we derived the top six high-scoring peptides from its sequence. These peptides were cloned into our uAb plasmids, and transfected into A 673 Ewing sarcoma cells, where 4E-BP2 is highly expressed. Following Western blotting post treatment, we successfully identified two degraders, 4E-BP2_SnP_3 and 4E-BP2_SnP_6, demonstrating over 50% degradation of endogenous 4E-BP2 as compared to that of a non-targeting control plasmid (FIG. 12a-b), highlighting the utility of our algorithm. We next turned our focus to TRIM8, an E3 ubiquitin ligase itself that regulates the levels of the core fusion oncoprotein driving Ewing sarcoma, EWS-FLI111. Loss of TRIM8 induces EWS-FLI1-mediated overdose in Ewing sarcoma cells, leading to upregulation of apoptosis. Using TRIM8 as an input into our curated PPI database to identify multiple interacting partners (FIG. 12A), we used SaLT&PepPr to derive the top six highest scoring peptides from various partners and integrated them into our uAb architecture. Next, we transfected these uAbs into A673 Ewing sarcoma cells, and successfully identified two candidates, TRIM8_SnP_5 and TRIM8_SnP_6, that degraded endogenous TRIM8 with statistical significance (FIG. 12C, FIG. 12D). We then co-transfected these six uAbs alongside a GFP-based fluorogenic caspase reporter of apoptosis in A673 cells, termed ZipGFP27, and observed that our most effective degraders induced upregulation of apoptosis, as expected from previous studies (FIG. 12E).

Further data is provided in “SaLT&PepPr is an interface-predicting language model for designing peptide-guided protein degraders” which can be found at https://doi.org/10.1038/s42003-023-05464-z., and which is herein incorporated by reference as if presented in its respective entirety. Furthermore, U.S. Provisional Application No. 63/344,820, entitled: “Contrastive Learning for Peptide Based Degrader Design and Uses Thereof” is also incorporated herein by reference as if presented in its entirety.

### Methods of Treatment

The present disclosure provides methods and compositions for the creation of engineered chimeras between a synthetic binding protein (e.g., antibodies, DARPins, FN3, monobodies, nanobodies, etc.) and a Post-Translational Modification (PTMs) domain—that have extended half-life inside of cells.

The present disclosure also provides a chimeric molecule in which the targeting domain is computationally designed.

The present disclosure further provides a chimeric molecule in which the targeting domain is computationally designed and is relatively non-homologous to wild type binders to said target (e.g. a non-natural sequence).

The present disclosure also provides a chimeric molecule in which the PTM domain is computationally designed (e.g. a computationally designed enzyme).

As used herein, the terms “chimeric molecule” or “ubiquibody” are used interchangeably and refer to a molecule possessing a degradation domain and a targeting domain, attached by a linker region, as defined herein.

As used herein, “deubiquitinating enzymes” or “DUBs” are enzymes that remove ubiquitin molecules from proteins in a process called deubiquitination. Ubiquitin is a small protein that is added to other proteins as a post-translational modification, and this modification can affect protein function, localization, and stability. DUBs play an important role in regulating the ubiquitin system by reversing the effects of ubiquitination. There are many different types of DUBs, each with unique characteristics and functions. Some DUBs remove ubiquitin from single ubiquitinated sites on a protein, while others can cleave entire chains of ubiquitin molecules. DUBs are involved in a wide range of cellular processes, including DNA repair, protein degradation, and immune response. Dysregulation of DUB activity has been linked to a number of diseases, including cancer, neurodegenerative disorders, and inflammatory diseases. In humans there are nearly 100 DUB genes, which can be classified into two main classes: cysteine proteases and metalloproteases. The cysteine proteases comprise ubiquitin-specific proteases (USPs), ubiquitin C-terminal hydrolases (UCHs), Machado-Josephin domain proteases (MJDs) and ovarian tumour proteases (OTU). The metalloprotease group contains only the Jab1/Mov34/Mpr1 Pad1 N-terminal+(MPN+) (JAMM) domain proteases.

Accordingly, one aspect of the present disclosure relates to a chimeric molecule comprising (i) a PTMs domains that comprises a degradation domain comprising a deubiquitinating enzymes and (ii) a targeting domain comprising a substrate-binding motif which is heterologous to the deubiquitinating enzyme. A linker couples the PTMs domain to the targeting domain.

In some embodiments of the compositions and methods according to the present disclosure, the chimeric molecule (or test agent) is an isolated chimeric molecule (or isolated test agent). As used herein, the terms “isolated” or “purified” polypeptide, peptide, molecule, or chimeric molecule, is substantially free of cellular material or other contaminating polypeptides from the cell or tissue source from which the agent is derived, or substantially free from chemical precursors or other chemicals when chemically synthesized. For example, a chimeric molecule would be free of materials that would interfere with such a molecule's intended function, diagnostic or therapeutic uses. Such interfering materials may include proteins or fragments other than the materials encompassed by the chimeric molecule, enzymes, hormones and other proteinaceous and nonproteinaceous solutes.

In some embodiments of the compositions and methods according to the present disclosure, the linker is heterologous to the PTMs domain and the targeting domain. In accordance with such embodiments, the linker is heterologous to both the PTMs motif of the degradation domain and the substrate-binding motif of the targeting domain.

As described herein, the substrate-binding motif of the targeting domain is heterologous to a PTMs domain. Accordingly, the PTMs domain may be heterologous to the targeting domain. Likewise, in some embodiments, the PTMs domain does not comprise a substrate-binding motif.

In one or more implementations, a peptide-based therapeutic is provided where the therapeutic includes any of the Peptide-E3 ubiquitin ligase or other polynucleotides described herein, or a sequence having 80% homology thereto. In one further implementation, the peptide therapeutic includes any of the foregoing polynucleotides coupled a delivery vector in which said delivery vector may be either a virus or micelle. Peptide-based therapeutic comprising the fusions of any of the foregoing polynucleotides in which said peptide fusion is further fused to a cell penetrating motif or a cell surface receptor binding motif. In certain embodiments, the compositions and methods of the present disclosure are useful for the prevention and/or treatment of symptoms of cancer and metastasis. In certain embodiments, the compositions and methods of the present disclosure are useful for the prevention and/or treatment of cancer and metastasis.

In one embodiment, the subject has a cancer and metastasis. In some embodiments, the cancer or metastasis is selected from the group of basal cell carcinoma (BCC), head and neck squamous cell carcinoma (HNSCC), prostate cancer (CaP), pilomatrixoma (PTR) and medulloblastoma (MDB).

Generation of plasmids. All uAb plasmids were generated from the standard pcDNA3 vector, harboring a cytomegalovirus (CMV) promoter and a C-terminal IRES-mCherry cassette. Target coding sequences (CDS) were synthesized as gBlocks from Integrated DNA Technologies (IDT). Sequences were amplified with overhangs for Gibson Assembly-mediated insertion into the pcDNA3-SARS-COV-2-S-RBD-sfGFP backbone (Addgene #141184) linearized by digestion with NheI and BamHI. An Esp3Irestriction site was introduced immediately upstream of the CHIPATPR CDS and flexible GSGSG linker via the KLD Enzyme Mix (NEB) following PCR amplification with mutagenic primers (Genewiz). For uAb assembly, oligos for candidate peptides were annealed and ligated via T4 DNA Ligase into the Esp3I-digested uAb backbone.

Assembled constructs were transformed into 50 μL NEB Turbo Competent Escherichia coli cells, and plated onto LB agar supplemented with the appropriate antibiotic for subsequent sequence verification of colonies and plasmid purification (Genewiz). For protein purification, genes encoding each of the uAb constructs were PCR amplified from pcDNA3-based plasmids using primers that introduced HindIII and Xhol overhangs. The resulting PCR amplicons were ligated in an empty pET28a vector, which had been doubly digested with/ndIII/Xhol. This process yielded plasmids which encoded each of the selected peptides followed by CHIPΔTPR, now bearing a 6xHis tag at its C-terminus. All plasmids were confirmed by DNA sequencing by Genewiz or at the Biotechnology Resource Center (BRC) Genomics Facility of the Cornell Institute of Biotechnology, and subjected to plasmid purification. Cell culture. The DLD1 cell line was a generous gift from Dr. Pengbo Zhou. DLD1 cells (ATCC CCL-221), HEK293T cells (ATCC CRL-3216), and A673 cells (ATCC CRL-1598) were cultured in DMEM supplemented with 100 units/mL penicillin, 100 mg/mL streptomycin, and 10% FBS. Unless otherwise noted, the day before the transfection, 0.3×106 cells were seeded in each well of a 6-well plate. uAb-expressing plasmids were prepared using the Pure Yield miniprep kit to remove endotoxins. On the day of transfection, plasmids were transfected by Lipofectamine 3000. After 3 days of incubation post-transfection, cell lysates were collected for immunoblotting. Cell fractionation and immunoblotting. For probing β-catenin in FIG. 2, on the day of harvest, cells were detached by addition of 0.05% trypsin-EDTA and cell pellets were washed twice with icecold 1×PBS. Cells were then lysed and subcellular fractions were isolated from lysates using a Subcellular Protein Fractionation Kit (ThermoFisher) per the manufacturer's instructions. Specifically, ice-cold cytosolic extraction buffer was added to the cell pellet, the mixture was placed at 4° C. for 10 min with gentle shaking followed by centrifugation at 500×g for 10 min at 4° C. The supernatant was collected immediately to a pre-chilled PCR tube and placed on ice followed by immunoblotting or stored at −20° C. for future usage. The pellet was then added with ice-cold membrane extraction buffer. The mixture was incubated at 4° C. for 10 min followed by centrifugation at 3000×g for 5 min. The supernatant was immediately transferred to a pre-chilled tube. Protein concentration was quantified using the Pierce BCA Protein Assay Kit (ThermoFisher). An equivalent amount of total protein was loaded into Precise Tris-HEPES 4-20% sodium dodecyl sulfate (SDS)-polyacrylamide gels (ThermoFisher) and separated by electrophoresis. Immunoblotting was performed according to standard protocols.

Briefly, proteins were transferred to poly(-vinylidene fluoride) (PVDF) membranes (Millipore), blocked with 5% (w/v) nonfat dry milk (Carnation) in 1×tris-buffered saline (TBS) with 0.05% (v/v) Tween 20 (TBST) at room temperature for 1 h, washed three times with TBST for 10 min, and probed with rabbit anti-β-catenin antibody (Cell Signaling, Cat #8480 S; diluted 1:1000) or rabbit anti-β-Tubulin (Cell Signaling Cat #2146; diluted 1:1000). The blots were washed again three times with TBST for 5 min each and then probed with a secondary antibody, donkey anti-rabbit-horseradish peroxidase (HRP) (Abcam, Cat #7083; diluted 1:2500), for 1 h at room temperature. Blots were detected by chemiluminescence using a ChemiDoc MP imager (Bio-Rad). Densitometry analysis of protein bands in immunoblots was performed using ImageJ software as described here: https://imagej.nih.gov/ij/docs/examples/dot-blot/. Briefly, bands in each lane were grouped as a row or a horizontal “lane” and quantified using ImageJ's gel analysis function. Intensity data for the uAb bands was normalized to band intensity for empty plasmid control cases from six independent experiments. For probing TRIM 8 and 4E-BP2 in FIG. 3, on the day of harvest, cells were detached by addition of 0.05% trypsin-EDTA and cell pellets were washed twice with ice-cold 1×PBS. Cells were then lysed and subcellular fractions were isolated from lysates using a 1:100 dilution of protease inhibitor cocktail (Millipore Sigma) in Pierce RIPA buffer (ThermoFisher). Specifically, the protease inhibitor cocktail-RIPA buffer solution was added to the cell pellet, the mixture was placed at 4° C. for 30 min followed by centrifugation at 15,000 rpm for 10 min at 4° C. The supernatant was collected immediately to a pre-chilled PCR tube, and after adding 4×Bolt™ LDS Sample Buffer (ThermoFisher) with 5% β-mercaptoethanol in a 3:1 ratio, the mixture was incubated at 95° C. for 10 min prior to immunoblotting. Immunoblotting was performed according to standard protocols. Briefly, samples were loaded at equal volumes into Bolt™ Bis-Tris Plus Mini Protein Gels (ThermoFisher) and separated by electrophoresis. iBlot™ 2 Transfer Stacks (Invitrogen) were used for membrane blot transfer, and following a 1 h room temperature incubation in SuperBlock™ Blocking Buffer (ThermoFisher), proteins were probed with rabbit anti-TRIM8 antibody (Cell Signaling, Cat #4936, diluted 1:500), rabbit anti-4EBP 2 antibody (Cell Signaling, Cat #2845 T, diluted 1:500), rabbit anti-Vinculin antibody (ThermoFisher, Cat #700062, diluted 1:500), or mouse anti-GAPDH (Santa Cruz Biotechnology, Cat #sc-47724; diluted 1:500) for overnight incubation at 4° C. The blots were washed three times with 1×TBST for 5 min each and then probed with a secondary antibody, goat anti-rabbit IgG (H+L), horseradish peroxidase (HRP) (ThermoFisher, Cat #31460, diluted 1:5000) or goat anti-mouse IgG (H+L) Poly-HRP COMMUNICATIONS BIOLOGY|https://doi.org/10.1038/s42003-023-05464-z ARTICLE COMMUNICATIONS BIOLOGY|(2023) 6:1081|https: //doi. org/10.1038/s 42003-023-05464-z|www.nature.com/commsbio 7 (ThermoFisher, Cat #32230, diluted 1:2000) for 1-2 h at room temperature. Following three washes with 1×TBST for 5 min each, blots were detected by chemiluminescence using an iBright 1500 Imaging System (ThermoFisher). Densitometry analysis of protein bands in immunoblots was performed using FIJI software as described here: https://imagej.nih.gov/ij/docs/examples/dotblot/. Briefly, bands in each lane were grouped as a row or a horizontal “lane” and quantified using FIJI's gel analysis function. Intensity data for the uAb bands was first normalized to band intensity of GAPDH (for TRIM 8) or vinculin (for 4E-BP2) in each lane then to the average band intensity for empty uAb vector control cases across replicates. TOPFlash assay. A total of 1×104 DLD1 cells were seeded on a white-bottom 96-well plate 20-24 h prior to transfection. On the day of transfection, each well received the following plasmids: M50 Super 8×TOPFlash plasmid (Addgene plasmid #12456) or M51 Super 8×FOPFlash (TOPFlash mutant; Addgene plasmid #12457), pCMV-Renilla29, and pcDNA3-SnP_7 or pcDNA3-SnP_8. A total of 100 ng of plasmid DNA in a ratio of TOPFlash/FOPFlash: Renilla:SnP7/SnP_8 uAb=1:0.1:3 was mixed with Lipofectamine 3000 reagent in serum free Opti-MEM medium and added dropwise to each well after incubation at room temperature for 15 min. After 48 h of incubation, cells were lysed and the firefly and Renilla luminescence signals were measured sequentially by the dual-luciferase reporter kit (Promega). Plates were read on a microplate reader (Tecan). The luciferase activities were measured and normalized against the control Renilla activities. Protein expression and purification. All purified uAb constructs, and unfused CHIPΔTPR were obtained from cultures of E. coliBL21(DE3) cells carrying pET28a-based plasmids encoding the SnP_7 the SnP_8 uAbs or CHIPΔTPR3.

Cells were grown in Luria-Bertani (LB) medium according to protocols described previously3. Briefly, protein expression was induced with 1 M isopropyl @-D-1-thiogalactopyranoside (IPTG) when the culture density, determined by optical density at 600 nm (OD600), reached 0.5-0.7 and proceeded for 12-16 h at 37° C. Following expression, cells were harvested by centrifugation at 10,000×g for 10 min at 4° C. The resulting pellets were resuspended in 10 mL of phosphate-buffered saline (PBS) and lysed using an EmulsiFlex-C5 high-pressure homogenizer (Avestin). Lysates were cleared of insoluble material by centrifugation at 10,000×g for 10 min at 4° C. Clarified lysates containing 6xHis-tagged proteins were subjected to gravity-flow Ni2+-affinity purification using HisPur Ni-NTA resin (ThermoFisher) following the manufacturer's protocols. Purified proteins were stored at 4° C. for up to 2 weeks. The final purity of all proteins was confirmed by Coomassie-blue staining of SDS-PAGE gels. ELISA. ELISA was performed according to previously published protocols 3. Briefly, 96-well plates (MaxiSorp; Nunc Nalgene) were incubated with 1 μg/mL of β-catenin (Biomatik, Cat #RPU 40704) diluted in PBS, pH 7.4, 50 μL/well, at 4° C. overnight. Plates were incubated with 200 μL blocking buffer (5% (w/v) nonfat dry milk (Carnation) in PBS) overnight at 4° C., then washed three times with 200 μL PBS-T (PBS, 0.1% (v/v) Tween 20) per well. Purified uAb constructs were biotinylated with EZ-Link™ NHS-Biotin (ThermoFisher, Cat #20217) following the manufacturer's instructions. The biotinylated uAb constructs were appropriately serially diluted in triplicate in PBS and added to the ELISA plates for 1 h at 37° C. Plates were washed three times with PBS-T, then incubated for 1 h at room temperature in the presence of HRPconjugated streptavidin (ThermoFisher, Cat #N 100; diluted 1:20,000), with shaking at 450 rpm. After another three PBS-T washes, 100 μL of 3,3′-5,5′-tetramethylbenzidine substrate (1-Step Ultra TMB-ELISA; ThermoFisher) was added to each well, and the plates were incubated at room temperature in the dark. The reaction was stopped by adding 100 μL of 2 M H2SO4, and absorbance was measured at a wavelength of 450 nm using a FilterMax F5 microplate spectrophotometer (Agilent). Proteomics. HEK293T cells were maintained in DMEM supplemented with 100 units/mL penicillin, 100 mg/mL streptomycin, and 10% FBS. Target-sfGFP (1 μg) and Target-sfGFP (1 μg) +pcDNA-uAb (1 μg) plasmids were transfected into cells as triplicates (8×104/well in a 6-well plate) with Lipofectamine 3000 (Invitrogen) in Opti-MEM (Gibco). Three days post transfection, cells were harvested and washed four times with 500 μL 1× cold PBS. The cell pellets were resuspended in 200 μL Pierce RIPA buffer (VWR) and incubated on ice for 15 min. The homogenates were treated with 20% (w/v) SDS in triethylammonium bicarbonate buffer, pH 8.5, followed by probe sonication and heating at 80° C. for 5 min. The supernatants were collected after centrifugation and the concentrations were determined using detergent-compatible Bradford assay. From each sample, 20 μg was reduced and alkylated, and digested with trypsin using an S-trap micro device. Peptide eluents were lyophilized, and after reconstitution, equal volumes of each sample were mixed to make an SPQC pool. Approximately 1 μg of each sample, and three replicates of the SPQC pool were analyzed by 1D-LCMS/MS. Samples were analyzed using a M-Class UPLC system (Waters) coupled to an Exploris 480 high resolution accurate tandem mass spectrometer (ThermoFisher) via a Nanospray Flex lon source and processed using Spectronaut 16. The p values were calculated by performing a Student's t-test on log2fc values. The log2fc values were calculated by the difference of average abundances of the proteins in the presence and absence of uAb. Functional assays. For the apoptosis assay, 3×105 A673 cells/well were seeded on a 24-well plate 20-24 h prior to transfection. On the day of transfection, each well received the following plasmids: ZipGFP-Casp3 plasmid (Addgene plasmid #81241) and pcDNA3-SnP_TRIM8_#. A total of 500 ng of plasmid DNA in a ratio of ZipGFP-Casp3:pcDNA 3-SnP_TRIM8 #=1:1 was mixed with Lipofectamine 2000 reagent in serum-free Opti-MEM medium and added dropwise to each well after incubation at room temperature for 20 min. After 60 h of incubation, cells were harvested and analyzed similarly as mentioned for uAb screening. Cells expressing mCherry were gated, and normalized EGFP cell fluorescence was calculated as compared to a sample transfected with a nontargeting uAb, using the FlowJo software (https://flowjo.com/).

To ensure robust reproducibility of all results, experiments were performed with at least three biological replicates and at least three technical measurements. Sample sizes were not predetermined based on statistical methods but were chosen according to the standards of the field (at least three independent biological replicates for each condition), which gave sufficient statistics for the effect sizes of interest. All data were reported as average values with error bars representing standard deviation (SD). For individual samples, unless described otherwise, statistical significance was determined by paired Student's t tests (*p<0.05, **p<0.01; ***p<0.001; ****p<0.0001). All graphs were generated using Prism 9 for MacOS version 9.2.0. No data were excluded from the analyses. The experiments were not randomized. The investigators were not blinded to allocation during experiments and outcome assessment.

### Pharmaceutical Compositions

The present disclosure thus provides pharmaceutical compositions that include Peptide-post translational modification fusion compounds and a pharmaceutically acceptable carrier. The compounds of the present disclosure can be formulated as pharmaceutical compositions and administered to a mammalian host, such as a human patient, in a variety of forms adapted to the chosen route of administration.

Routes of administration include, but are not limited to oral, topical, mucosal, nasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural and sublingual administration.

As used herein, the term “administering” generally refers to any and all means of introducing compounds described herein to the host subject. Compounds described herein may be administered in unit dosage forms and/or compositions containing one or more pharmaceutically-acceptable carriers, adjuvants, diluents, excipients, and/or vehicles, and combinations thereof.

As used herein, the terms “composition” generally refers to any product comprising more than one ingredient, including the compounds described herein. It is to be understood that the compositions described herein may be prepared from compounds described herein or from salts, solutions, hydrates, solvates, and other forms of the compounds described herein. It is appreciated that the compositions may be prepared from various amorphous, non-amorphous, partially crystalline, crystalline, and/or other morphological forms of the compounds described herein, and the compositions may be prepared from various hydrates and/or solvates of the compounds described herein. Accordingly, such pharmaceutical compositions that recite compounds described herein include each of, or any combination of, or individual forms of, the various morphological forms and/or solvate or hydrate forms of the compounds described herein.

In some embodiments, the Peptide-post translational modification fusion based treatments may be systemically (e.g., orally) administered in combination with a pharmaceutically acceptable vehicle such as an inert diluent or an assimilable edible carrier. For oral therapeutic administration, the active compound may be combined with one or more excipients and used in the form of ingestible tablets, buccal tablets, sublingual tablets, troches, capsules, elixirs, suspensions, syrups, wafers, and the like. The percentage of the compositions and preparations may vary and may be between about 1 to about 99% weight of the active ingredient(s) and excipients such as, but not limited to a binder, a filler, a diluent, a disintegrating agent, a lubricant, a surfactant, a sweetening agent; a flavoring agent, a colorant, a buffering agent, anti-oxidants, a preservative, chelating agents (e.g., ethylenediaminetetraacetic acid), and agents for the adjustment of tonicity such as sodium chloride.

Suitable binders include, but are not limited to, polyvinylpyrrolidone, copovidone, hydroxypropyl methylcellulose, starch, and gelatin.

Suitable fillers include, but are not limited to, sugars such as lactose, sucrose, mannitol or sorbitol and derivatives therefore (e.g. amino sugars), ethylcellulose, microcrystalline cellulose, and silicified microcrystalline cellulose.

Suitable diluents include, but are not limited to, dicalcium phosphate dihydrate, sugars, lactose, calcium phosphate, cellulose, kaolin, mannitol, sodium chloride, and dry starch.

Suitable disintegrants include, but are not limited to, pregelatinized starch, crospovidone, crosslinked sodium carboxymethyl cellulose and combinations thereof.

Suitable lubricants include, but are not limited to, sodium stearyl fumarate, stearic acid, polyethylene glycol or stearates, such as magnesium stearate.

Suitable surfactants or emulsifiers include, but are not limited to, polyvinyl alcohol (PVA), polysorbate, polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil.

Suitable flavoring agents and sweeteners include, but are not limited to, sweeteners such as sucralose and synthetic flavor oils and flavoring aromatics, natural oils, extracts from plants, leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot.

Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

Suitable buffering or pH adjusting agent include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide and magnesium hydroxide.

Suitable tonicity enhancing agents include, but are not limited to, ionic and non-ionic agents such as, alkali metal or alkaline earth metal halides, urea, glycerol, sorbitol, mannitol, propylene glycol, and dextrose.

Suitable wetting agents include, but are not limited to, glycerin, cetyl alcohol, and glycerol monostearate.

Suitable preservatives include, but are not limited to, benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide.

Suitable antioxidants include, but are not limited to, sorbic acid, ascorbic acid, ascorbate, glycine, α-tocopherol, butylated hydroxyanisole (BHA), and butylated hydroxytoluene (BHT).

The Peptide-post translational modification fusion based treatments of the present disclosure may also be administered via infusion or injection (e.g., using needle (including microneedle) injectors and/or needle-free injectors). Solutions of the active composition can be aqueous, optionally mixed with a nontoxic surfactant and/or may contain carriers or excipients such as salts, carbohydrates and buffering agents (preferably at a pH of from 3 to 9), and, for some applications, they may be more suitably formulated as a sterile non-aqueous solution or as a dried form to be used in conjunction with a suitable vehicle such as sterile, pyrogen-free water or phosphate-buffered saline. For example, dispersions can be prepared in glycerol, liquid polyethylene glycols, triacetin, and mixtures thereof and in oils. The preparations may further contain a preservative to prevent the growth of microorganisms.

The pharmaceutical compositions may be formulated for parenteral administration (e.g., subcutaneous, intravenous, intra-arterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Water is a preferred carrier when the pharmaceutical composition is administered intravenously. Saline solutions and aqueous dextrose and glycerol solutions can also be employed as liquid carriers, particularly for injectable solutions. Oils such as petroleum, animal, vegetable, or synthetic oils and soaps such as fatty alkali metal, ammonium, and triethanolamine salts, and suitable detergents may also be used for parenteral administration. Further, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. Suitable preservatives include e.g. sodium benzoate, benzoic acid, and sorbic acid. Suitable antioxidants include e.g. sulfites, ascorbic acid and ⊏-tocopherol.

The preparation of parenteral compounds/compositions under sterile conditions, for example, by lyophilization, may readily be accomplished using standard pharmaceutical techniques well known to those skilled in the art.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. In one embodiment, the compositions are administered by the oral or nasal respiratory route for local or systemic effect. Compositions in pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, orally or nasally, from devices that deliver the formulation in an appropriate manner.

In yet another embodiment, the composition is prepared for topical administration, e.g. as an ointment, a gel, a drop or a cream. For topical administration to body surfaces using, for example, creams, gels, drops, ointments and the like, the compounds of the present disclosure can be prepared and applied in a physiologically acceptable diluent with or without a pharmaceutical carrier. Adjuvants for topical or gel base forms may include, for example, sodium carboxymethylcellulose, polyacrylates, polyoxyethylene-polyoxypropylene-block polymers, polyethylene glycol and wood wax alcohols.

Alternative formulations include nasal sprays, liposomal formulations, slow-release formulations, pumps delivering the drugs into the body (including mechanical or osmotic pumps) controlled-release formulations and the like, as are known in the art.

### Doses

As used herein, the term “therapeutically effective dose” means (unless specifically stated otherwise) a quantity of a compound which, when administered either one time or over the course of a treatment cycle affects the health, wellbeing or mortality of a subject.

A Peptide-post translational modification fusion based treatment described herein can be present in a composition in an amount of about 0.001 mg, about 0.005 mg, about 0.01 mg, about 0.02 mg, about 0.03 mg, about 0.04 mg, about 0.05 mg, about 0.06 mg, about 0.07 mg, about 0.08 mg, about 0.09 mg about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 0.5 mg, about 10 mg, about 10.5 mg, about 11 mg, about 12 mg, about 12.5 mg, about 13 mg, about 13.5 mg, about 14 mg, about 14.5g, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg.

A Peptide-post translational modification fusion based treatment described herein described herein can be present in a composition in a range of from about 0.1 mg to about 100 mg; 0.1 mg to about 75 mg; from about 0.1 mg to about 50 mg; from about 0.1 mg to about 25 mg; from about 0.1 mg to about 10 mg; 0.1 mg to about 7.5 mg, 0.1 mg to about 5 mg; 0.1 mg to about 2.5 mg; from about 0.1 mg to about 1 mg; from about 0.5 mg to about 100 mg; from about 0.5 mg to about 75 mg; from about 0.5 mg to about 50 mg; from about 0.5 mg to about 25 mg; from about 0.5 mg to about 10 mg; from about 0.5 mg to about 5 mg, from about 0.5 mg to about 2.5 mg; from about 0.5 mg to about 1 mg; from about 1 mg to about 100 mg; from about 1 mg to about 75 mg; from about 0.1 mg to about 50 mg; from about 0.1 mg to about 25 mg; from about 0.1 mg to about 10 mg; from about 0.1 mg to about 5 mg; from about 0.1 mg to about 2.5 mg; from about 0.1 mg to about 1 mg.

### Dosing Regimens

The compounds described herein can be administered by any dosing schedule or dosing regimen as applicable to the patient and/or the condition being treated. Administration can be once a day (q.d.), twice a day (b.i.d.), thrice a day (t.i.d.), once a week, twice a week, three times a week, once every 2 weeks, once every three weeks, or once a month twice, and the like.

In some embodiments, the Peptide-post translational modification fusion based treatment is administered for a period of at least one day. In other embodiments, the Peptide-post translational modification fusion based treatment is administered for a period of at least 2 days. In other embodiments, the Peptide-post translational modification fusion based treatment is administered for a period of at least 3 days. In other embodiments, the Peptide-post translational modification fusion based treatment is administered for a period of at least 4 days. In other embodiments, the Peptide-post translational modification fusion based treatment is administered for a period of at least 5 days. In other embodiments, the Peptide-post translational modification fusion based treatment is administered for a period of at least 6 days. In other embodiments, the Peptide-post translational modification fusion based treatment is administered for a period of at least 7 days. In other embodiments, the Peptide-post translational modification fusion based treatment is administered for a period of at least 10 days. In other embodiments, the Peptide-post translational modification fusion based treatment is administered for a period of at least 14 days. In other embodiments, the Peptide-post translational modification fusion based treatment is administered for a period of at least one month. In some embodiments, the Peptide-post translational modification fusion based treatment is administered chronically for as long as the treatment is needed.

The present subject matter described herein will be illustrated more specifically by the following non-limiting examples, it being understood that changes and variations can be made therein without deviating from the scope and the spirit of the disclosure as hereinafter claimed. It is also understood that various theories as to why the disclosure works are not intended to be limiting.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments, but should be defined only in accordance with the following claims and their equivalents.

For example, the following implementations are understood.

Implementation 1, a Method of generating binding peptide sequences to a target sequence, the method comprising:
a. Receiving, using a processor configured by code executing therein, a data object corresponding to a protein target;
b. Searching, using the data object, a protein interaction database for at least one partner protein to the target protein;
c. Identifying at least one partner protein to the target protein;
d. Providing the at least one partner protein to a computational model configured to output a predicted protein sequence predicted to interact with the target sequence; and

Identifying at least one subsequence within the predicted protein sequence that meets a predetermined interaction threshold. The method of any preceding implementation, further comprising:
a. Converting the identified at least one partner protein into a FASTA data format prior to providing the at least one partner protein to the computational model.

The method of any preceding implementation, wherein the computation model is a protein language model.

The method of any preceding implementation, wherein the protein language model is configured to generate predicted protein sequences without the use structural data relating to the target protein.

The method of any preceding implementation, wherein the computation model is a least a 500-million parameter protein language model.

The method of any preceding implementation, wherein the computation model further includes a multilayer perceptron classification head configured to receive the output of the protein language model.

The method of any preceding implementation, wherein identifying at least one subsequence includes using receiving a user-specified length for a target binding generated sequence and using greedy sampling to generate subsequences of the predicted protein sequence based on the highest predicted probability of binding to the target.

An implementation where a pre-trained protein language model, that when executed by a processor, configures the processor to generate one or more guide peptide sequences without the use of structural data.

An implementation of a method for training a machine learning model to predict the cosine similarity between two protein sequences, comprising the steps of: generating a matrix of all possible pairs of target and peptide sequences; generating an embedding for each sequence in the matrix using a protein language model; calculating the cosine similarity between each pair of embeddings in the matrix; calculating the cross-entropy loss between the predicted cosine similarities and the actual cosine similarities; averaging the cross-entropy losses of the matrix; updating the model parameters to minimize the average cross-entropy loss.

The method of any preceding implementation, further comprising generating a training dataset comprised of experimentally validated protein-protein interactions and training the machine learning model.

What is claimed is:

1. A Method of generating binding peptide sequences to a target sequence, the method comprising:
a. Receiving, using a processor configured by code executing therein, a data object corresponding to a protein target;
b. Searching, using the data object, a protein interaction database for at least one partner protein to the target protein;
c. Identifying at least one partner protein to the target protein;
d. Providing the at least one partner protein to a computational model configured to output a predicted protein sequence predicted to interact with the target sequence; and
e. Identifying at least one subsequence within the predicted protein sequence that meets a predetermined interaction threshold.

2. The method of claim 1, further comprising:
a. Converting the identified at least one partner protein into a FASTA data format prior to providing the at least one partner protein to the computational model.

3. The method of claim 1, wherein the computation model is a protein language model.

4. The method of claim 3, wherein the protein language model is configured to generate predicted protein sequences without the use structural data relating to the target protein.

5. The method of claim 3, wherein the computation model is a least a 500-million parameter protein language model.

6. The method of claim 3, wherein the computation model further includes a multilayer perceptron classification head configured to receive the output of the protein language model.

7. The method of claim 1, wherein identifying at least one subsequence includes using receiving a user-specified length for a target binding generated sequence and using greedy sampling to generate subsequences of the predicted protein sequence based on the highest predicted probability of binding to the target.

8. A pre-trained predictive model configured to be executed as code by at least one processor, wherein the predictive model is configured to receive one or more sequences input data and generate one or more guide peptide sequences without the use of structural data, wherein the predictive model includes a pre-trained protein language configured to output data to a four layer fully connected neural network classification head.

9. The pre-trained predicative model of claim 8, wherein the neural network classification head is configured to receive output data from the protein language model generate a predicted interaction likelihood for each amino acid.

10. A method for training a machine learning model to predict the cosine similarity between two protein sequences, comprising the steps of: generating a matrix of all possible pairs of target and peptide sequences; generating an embedding for each sequence in the matrix using a protein language model; calculating the cosine similarity between each pair of embeddings in the matrix; calculating the cross-entropy loss between the predicted cosine similarities and the actual cosine similarities; averaging the cross-entropy losses of the matrix; updating the model parameters to minimize the average cross-entropy loss.

11. The method of claim 1, further comprising generating a training dataset comprised of experimentally validated protein-protein interactions and training the machine learning model.
